(19) United States
(12) Patent Application Publication
(10) Pub. No.: US 2018/0362765 A1
(43) Pub. Date: Dec. 20, 2018

(54) FIBER ASSEMBLY AND METHOD FOR MANUFACTURING THE SAME

(71) Applicant: Panasonic Intellectual Property Management Co., Ltd., Osaka (JP)

(72) Inventors: Hirokazu KIMIYA, Kyoto (JP); Tomoko KAWASHIMA, Osaka (JP)

(21) Appl. No.: 15/986,906

(22) Filed: May 23, 2018

(30) Foreign Application Priority Data
Jun. 15, 2017 (JP) 2017-117993

(51) Int. Cl. C08L 89/00; A61L 15/60; A61L 15/22; A61L 15/42; A61L 26/00; A61L 15/44; A61K 8/02; A61K 8/73; A61K 8/65; A61K 9/00; A61K 9/70; A61K 47/42; A61K 47/36; D01D 5/00

(52) U.S. Cl. CPC C08L 89/00; C08L 2205/16; A61L 15/225; A61L 15/42; A61L 26/008; A61L 26/0061; A61L 26/0066; A61L 15/44; A61L 26/0023; A61L 26/0033; A61L 26/0052; A61K 8/0216; A61K 8/733; A61K 8/735; A61K 8/65; A61K 9/0014; A61K 9/7007; A61K 47/42; A61K 47/36; D01D 5/0046; A61L 15/60

(57) ABSTRACT

To easily manufacture a fiber assembly that includes fibers and particulates supported by the fibers, provided is a method for manufacturing a fiber assembly including: a preparation step of preparing a raw material liquid that contains a water-soluble first component, a second component that is capable of forming a hydrogel, and water; and an electrospinning step of forming fibers that contain the first component as a main component and particulates that are supported by a plurality of the fibers and contain the second component from the raw material liquid by an electrospinning method, wherein in a case where the fibers contain the second component, a mass proportion of the second component contained in the particulates is greater than a mass proportion of the second component contained in the fibers.

### TECHNICAL FIELD

The present invention relates to a fiber assembly and a method for manufacturing the same, and more particularly to a fiber assembly that includes fibers and particulates supported by the fibers.

### BACKGROUND ART

In recent years, a method has been proposed in which a functional component such as collagen is retained in advance in a sheet-like material that is used in contact with the skin. For example, Patent Literature 1 teaches a sheet-like material in which capsules encapsulating collagen are carried on a fiber layer.

### CITATION LIST

### Patent Literature

[PTL 1] Laid-Open Patent Publication No. 2014-129314

### SUMMARY OF INVENTION

### Technical Problem

According to Patent Literature 1, the capsules encapsulating collagen are sprayed to nanofibers produced by electrospinning so as to attach the capsules to the outer surface of the nanofibers. In this case, the capsules are easily detached from the nanofibers. In addition, Patent Literature 1 also teaches a method in which capsules encapsulating collagen are mixed with a raw material for nanofibers, and the resulting mixture is electrospun so as to incorporate the capsules in the nanofibers. In this case, the detachment of the capsules is suppressed, but it is difficult to obtain a sufficient effect produced by the use of collage because the capsules have a small exposed area. Furthermore, with this method, it is necessary to separately prepare the capsules encapsulating collagen, and thus the productivity is likely to decrease.

Besides the above, nanofibers that carry various functional components are proposed. However, because nanofibers are fine and have a small volume, it is not possible to cause nanofibers to carry sufficient amounts of functional components. For this reason, it is difficult to obtain a sufficient effect produced by the use of a functional component.

### Solution to Problem

One aspect of the present invention relates to a method for manufacturing a fiber assembly including: a preparation step of preparing a raw material liquid that contains a water-soluble first component, a second component that is capable of forming a hydrogel, and water; and an electrospinning step of forming fibers that contain the first component as a main component and particulates that are supported by a plurality of the fibers and contain the second component from the raw material liquid by an electrospinning method, wherein in a case where the fibers contain the second component, a mass proportion of the second component contained in the particulates is greater than a mass proportion of the second component contained in the fibers.

Another aspect of the present invention relates to a fiber assembly including: fibers that contain a water-soluble first component as a main component; and particulates that contain a second component that is capable of forming a hydrogel, wherein at least a portion of the particulates are supported by a plurality of the fibers, and in a case where the fibers contain the second component, a mass proportion of the second component contained in the particulates is greater than a mass proportion of the second component contained in the fibers.

### Advantageous Effects of Invention

According to the method for manufacturing a fiber assembly according to the present invention, it is possible to easily manufacture a fiber assembly that includes fibers and particulates that are supported by the fibers and contain a large amount of a second component that is capable of forming a hydrogel. Also, with the fiber assembly according to the present invention, it is possible to increase the exposed area of the particulates while suppressing detachment of the particulates.

### BRIEF DESCRIPTION OF DRAWINGS

FIG. 1 is a side view showing an example of a configuration of an electrospinning apparatus that is used to manufacture a fiber assembly according to one embodiment of the present invention.

FIG. 2 is an electron micrograph of a fiber assembly obtained in Example 1 (at a magnification of 5,000 times).

FIG. 3 is an infrared absorption spectrum of the fiber assembly obtained in Example 1.

FIG. 4 is an infrared absorption spectrum of particulates obtained in Example 1.

FIG. 5 is an infrared absorption spectrum of enzyme-degraded collagen peptide.

FIG. 6 is an infrared absorption spectrum of sodium hyaluronate.

FIG. 7 is a graph showing a relationship between the mass proportion of sodium hyaluronate and the peak intensity ratio.

FIG. 8A is an electron micrograph of a fiber assembly obtained in Example 2 (at a magnification of 10,000 times).

FIG. 8B is an enlarged electron micrograph of FIG. 8A (at a magnification of 50,000 times)

FIG. 9 is an electron micrograph of a fiber assembly obtained in Example 3 (at a magnification of 5,000 times).

FIG. 10 is an electron micrograph of a fiber assembly obtained in Example 4 (at a magnification of 5,000 times).

### DESCRIPTION OF EMBODIMENT

### (Method for Manufacturing Fiber Assembly)

A method for manufacturing a fiber assembly according to the present embodiment includes: a preparation step of preparing a raw material liquid that contains a water-soluble first component, a second component that is capable of forming a hydrogel, and water; and an electrospinning step of forming fibers that contain the first component as a main component and particulates that are supported by a plurality of the fibers and contain the second component from the raw material liquid by an electrospinning method.

In an electrospinning method, a target is prepared and grounded or negatively (or positively) charged, and a raw material liquid (normally, a solution in which a raw material for fibers are dissolved) to which a positive (or negative) potential has been applied is discharged toward the target through a nozzle. The solvent contained in the raw material liquid is volatilized before it reaches the target, and an assembly of fibers produced by an electrostatic drawing phenomenon is deposited on the target.

Here, the raw material liquid used in the present embodiment contains a water-soluble first component, a second component that forms a hydrogel, and water. When the raw material liquid is electrostatically drawn, at least a portion of water contained in the raw material liquid is removed (evaporated). As a result, the first component forms fibers. At this time, the viscosity of the raw material liquid may be increased due to the inclusion of the second component. For this reason, the fibers are easily formed. During this process, a portion of the second component is introduced into the fibers. On the other hand, the concentration of the second component is increased, and the second component forms a gel structure and turns into particulates as a result of being released from the discharge pressure. During this process, a portion of the first component may be introduced into the gel structure. However, in the case where the fibers contain the second component, the mass proportion R_{2P} of the second component contained in the particulates is greater than the mass proportion R_{2F} of the second component contained in the fibers.

As described above, because a plurality of fibers containing the first component and a plurality of particulates containing the second component are produced within the same space, the plurality of fibers and the plurality of particulates come into contact with and bond to each other. After that, the plurality of fibers that support the particulates are deposited on the target, and a fiber assembly is formed. Furthermore, as a result of the above-described process being performed continuously, the particulates are bonded to one or more fibers at a plurality of contact points. Accordingly, detachment of the particulates is further suppressed. The difference in spinnability between the first component and the second component is considered to result from the differences in the molecular weight, the solubility in solvents, the surface tension, the intermolecular interaction, and the like.

The fiber assembly manufactured by the method described above includes: fibers that contain a water-soluble first component as a main component; and particulates that contain a second component that is capable of forming a hydrogel. The fiber assembly may be in the form of, for example, a non-woven fabric or cotton.

Hereinafter, the steps of the method for manufacturing a fiber assembly according to the present embodiment, and the configuration of the fiber assembly will be described specifically by way of an embodiment suitable for use as a sheet for application to the skin that is used in direct or indirect contact with the skin or by being attached to the skin. However, the application and configuration of the fiber assembly is not limited thereto. For example, the first component and the second component may be selected as appropriate according to the application or the like of the fiber assembly.

### (Preparation Step)

First, a raw material liquid 20 that contains a first component, a second component and water is prepared.

The first component is water soluble and is dissolved in the raw material liquid 20. Also, the first component is a component that does not form a hydrogel. The first component may be, for example, any of collagens. Examples of the collagens include collagen, collagen peptide, gelatin, and the like. From the viewpoint of providing water solubility and ease of forming fibers, the first component preferably has a weight average molecular weight of 500 to 80,000, and more preferably 1,000 to 40,000.

The second component is a component that is capable of forming a hydrogel, and is dissolved or dispersed in the raw material liquid 20. The second component described above may be, for example, at least one selected from the group consisting of a hyaluronic acid salt, a hyaluronic acid derivative, a water-soluble alginic acid salt, and an alginic acid derivative. Examples of cations that form salts include sodium ions, potassium ions, magnesium ions, ammonium ions, calcium ions, and the like. Note, however, that alginic acid salts formed by divalent cations (for example, calcium ions) other than magnesium ions are not included in the second component because they are not water soluble. Examples of derivatives include esters, acetylated products, and the like. The second component may be used singly or in a combination of two or more.

Among these, from the viewpoint of providing a moisturizing effect, the second component is preferably a hyaluronic acid salt. As a result of a hyaluronic acid salt being contained as the second component, it is possible to impart a moisturizing effect to the fiber assembly. Also, from the viewpoint of providing a hemostasis effect, the second component is preferably a salt formed by calcium ions. As a result of a calcium salt being contained as the second component, it is possible to impart a hemostasis effect to the fiber assembly.

The concentration of the first component in the raw material liquid 20 is not particularly limited and may be set as appropriate by taking into consideration the viscosity of the raw material liquid 20, or the like. In terms of the formability of fibers, the concentration of the first component in the raw material liquid 20 is preferably 1 to 40 mass %, and more preferably 5 to 30 mass %. The concentration of the second component in the raw material liquid 20 is not particularly limited and may be set as appropriate as long as the concentration is within a range that does not cause the raw material liquid 20 to be gelled. In particular, from the viewpoint of ease of forming particulates, the concentration of the second component in the raw material liquid 20 is preferably 0.01 to 5 mass %, and more preferably 0.1 to 2 mass %.

The viscosity of the raw material liquid 20 may be set as appropriate so as to be suitable for the electrospinning method. In particular, the raw material liquid 20 preferably has a viscosity of 500 to 30,000 m Pa·s, and more preferably 1,000 to 15,000 m Pa·s. The viscosity is measured under conditions at 25° C. with the use of a rotational viscometer at a shear rate of 1 s⁻¹. When the raw material liquid 20 has a viscosity within the above-described range, stable electrospinning is possible, and the particulates are uniformly laid in the fiber assembly with ease. As described above, because the second component has the effect of increasing the viscosity of the raw material liquid 20, the viscosity of the raw material liquid 20 can be controlled by blending the second component. However, the raw material liquid 20 may contain other components for adjusting the viscosity.

The raw material liquid 20 may contain a solvent (hereinafter, referred to as “second solvent”) other than water. There is no particular limitation on the second solvent as long as it is compatible with water. The second solvent may be selected as appropriate according to the types of the first component and the second component, the manufacturing conditions, and the like. In particular, from the viewpoint of excellent compatibility with water and excellent volatility, the second solvent is preferably any of alcohols including methanol, ethanol, 1-propanol, 2-propanol, isobutyl alcohol, and hexafluoro isopropanol. These may be used singly or in a combination of two or more. However, from the viewpoint of ensuring solubility of the first component, the proportion of the second solvent in the total amount of the solvents is preferably less than 50 mass %, and more preferably less than 25 mass %.

The raw material liquid 20 may contain a functional component (third component) other than the first component (for example, any of collagens) and the second component (for example, at least one selected from the group consisting of a hyaluronic acid salt, a hyaluronic acid derivative, a water-soluble alginic acid salt, and an alginic acid derivative).

In this case, the third component is contained in at least either of the fibers and the particulates. According to the present embodiment, with a very simple operation of blending a functional component in the raw material liquid 20, it is possible to cause the fiber assembly to retain various types of functional components in a less detachable manner.

The third component may be water soluble, less water soluble, or water dispersible. Also, the third component may be a substance that is capable of forming a hydrogel, or a substance that does not form a hydrogel. The third component may be, for example, a pharmaceutical component that has a medicinal effect, a cosmetic component that is expected to provide a cosmetic effect, or an adjusting component that adjusts the properties of the raw material liquid 20, or the like. Examples of the pharmaceutical component include a hemostatic agent, an antiphlogistic agent, an autoinducer inhibitor, a transdermal pharmaceutical product, and the like. Examples of the cosmetic component include a vitamin C derivative, lactic acid, malic acid, a malic acid salt or derivative, tartaric acid, a tartaric acid salt or derivative, citric acid, a citric acid salt or derivative, sericin, a perfume, and the like. Examples of the adjusting component include a thickener, an antiseptic agent, a pH adjusting agent, an electroconductivity adjusting agent, and the like. The third component may be contained singly or in a combination of two or more

The third component may be incorporated more in the fibers, or may be incorporated more in the particulates depending on the level of compatibility with the first component and the second component, the solubility in water, or the like. For example, a third component that is highly compatible with the first component is likely to be incorporated in the fibers. It is preferable that a pharmaceutical component and/or a cosmetic component are/is incorporated in the fibers as the third component because the component(s) can act on the skin in a short time by dissolution of the first component. The dissolution of the first component is controlled by adjusting, for example, the supply of moisture or the humidity of the surroundings.

On the other hand, a third component that is highly compatible with the second component is likely to be incorporated in the particulates. It is preferable that a pharmaceutical component and/or a cosmetic component are/is incorporated in the particulates as the third component because the component(s) can act on the skin over a long period of time.

In particular, the raw material liquid 20 preferably contains a pH adjusting agent as the third component. It is thereby possible to easily adjust the viscosity of the raw material liquid 20 to a viscosity level suitable for spinning. This is because the solubility of a collagen, a hyaluronic acid salt and a water-soluble alginic acid salt in water is dependent on the pH level. Also, as a result of the raw material liquid 20 containing a pH adjusting agent, the pH level of the fibers and/or the particulates can be controlled. Here, the performance of the function of the pharmaceutical component and the cosmetic component may be dependent on the pH level. For this reason, in the case where the fibers and/or the particulates contain at least one of a pharmaceutical component and a cosmetic component as the third component, by further adding a pH adjusting agent as the third component, the effect of the pharmaceutical component and/or the cosmetic component is more easily exerted.

Examples of the pH adjusting agent include: acids such as citric acid, acetic acid, phosphoric acid, sulfuric acid, gluconic acid, and succinic acid; carbonates such as potassium carbonate and sodium hydrogencarbonate; sodium hydroxide; potassium hydroxide; and the like. It is preferable that the components listed above are used in the form of a buffer solution that contains a salt thereof (for example, a phosphoric acid buffer solution, a citric acid buffer solution, an acetic acid buffer solution, or the like) so as to stabilize the pH level.

There is no particular limitation on the concentration of the third component in the raw material liquid 20 as long as the concentration is within a range that does not hinder the formation of fibers and particulates. The concentration of the third component may be set as appropriate by taking into consideration the function of the third component. The concentration of the third component is preferably, for example, 0.01 to 5 mass %, and more preferably 0.1 to 2 mass %.

### (Electrospinning Step)

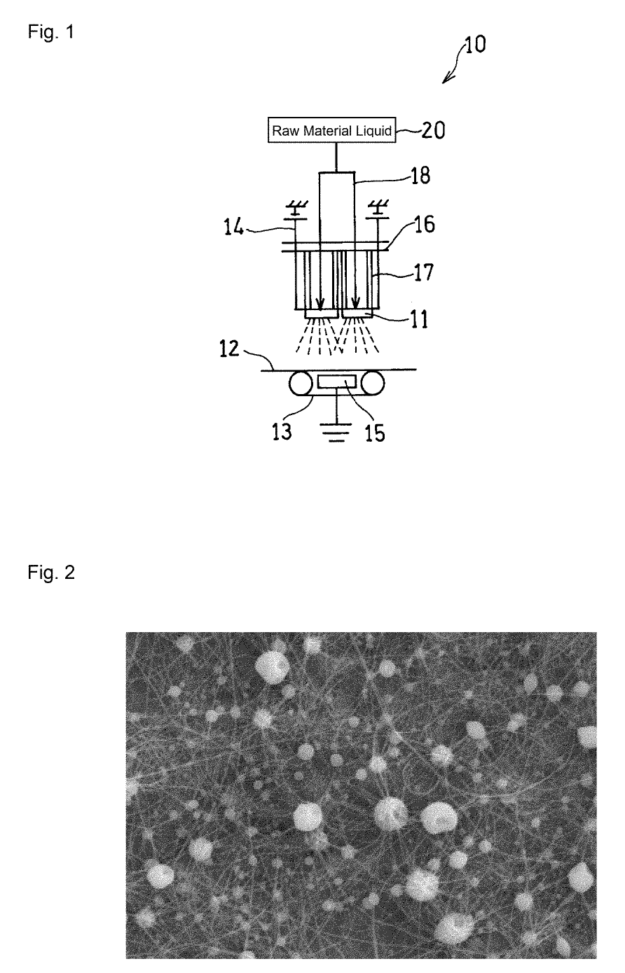

An electrospinning apparatus used in electrospinning will be described with reference to the drawings. FIG. 1 is a side view showing an example of a configuration of an electrospinning apparatus 10. The electrospinning apparatus 10 includes, for example, discharging units 11 for discharging a raw material liquid 20, a charging means that positively charges the discharged raw material liquid 20, and a conveyor belt 13 that supports a target 12. The conveyor belt 13 functions, together with the target 12, as a collector unit that collects a fiber assembly.

Each discharging unit 11 is made of a conductor, has an elongated shape, and is internally provided with a hollow portion. The hollow portion serves as a housing portion that houses the raw material liquid 20. A plurality of discharge outlets (not shown) for discharging the raw material liquid 20 are provided in a plurality of locations on the side of the discharging unit 11 that opposes the target 12. The distance between the discharge outlets of the discharging unit 11 and the target 12 may be, for example, 100 to 600 mm although it depends on the scale of the electrospinning apparatus 10 and the desired fiber diameter.

The raw material liquid 20 is supplied to the hollow portions of the discharging units 11 through pipes 18 by the pressure of a pump (not shown) that is in communication with the hollow portions of the discharging units 11, and discharged toward the target 12 through the discharge outlets. The discharged raw material liquid 20 in a charged state causes an electrostatic explosion while moving through a space (production space) between the discharging units 11 and the target 12 so as to produce fibers that contain the first component and particulates that contain the second component. The produced fibers and the particulates supported by the fibers are deposited on the target 12, thereby forming a fiber assembly. The amount of deposited fibers and the average fiber diameter D1 of the fibers are controlled by adjusting the pressure at which the raw material liquid 20 is discharged, the applied voltage, the composition of the raw material of the raw material liquid 20, the concentration of the raw material of the raw material liquid 20, and the environment (environmental composition, temperature, humidity, pressure and the like) of the production space.

The charging means for charging the discharging units 11 and the target 12 are constituted by a voltage application apparatus 14 for applying voltage to the discharging units 11 and a counter electrode 15 that is provided in parallel to the conveyor belt 13. The counter electrode 15 is earthed (grounded). Accordingly, a potential difference that corresponds to the voltage applied by the voltage application apparatus 14 can be generated between the discharging units 11 and the counter electrode 15 (the target 12). There is no particular limitation on the configuration of the charging means. For example, the target 12 may be negatively charged. Also, instead of providing the counter electrode 15, the conveyor belt 13 may be made by using a conductor.

Above the discharging units 11, a first supporting unit 16 parallel to the target 12 is installed. The discharging units 11 are supported by, for example, a second supporting unit 17 extending downward from the first supporting unit 16 such that the longitudinal direction of the discharging units 11 is parallel to the main surface of the target 12. The first supporting unit 16 may be movable such that it can pivotally move the discharging unit 11.

The electrospinning apparatus 10 is not limited to the configuration described above. For example, each discharging unit 11 may have a cross section whose shape gradually tapers from its upper end toward its lower end (V-shaped nozzle), the cross section being perpendicular to the longitudinal direction of the discharging unit 11. Also, the discharging unit 11 may include one or more needle shaped nozzles.

After the electrospinning step, the adjustment of water content or the removal of the solvents contained in the fibers and/or the particulates may be performed by air drying, decompression, or heating under conditions that do not cause damage to the fiber assembly. The water content may affect, in addition to the softness and texture of the fiber assembly, the action of each component, the storage properties of the fiber assembly, and the like.

### (Fiber Assembly)

The fiber assembly includes fibers that contain a water-soluble first component as a main component, and particulates that contain a second component that is capable of forming a hydrogel as a main component. At least a portion of the particulates is supported by a plurality of fibers that were described above.

### (Fibers)

The fibers function to support the particulates. Furthermore, under the presence of moisture, the fibers can also act on the skin. This is because the fibers contain a water-soluble first component as a main component (the component that accounts for 50 mass % or more of the fibers). When the fiber assembly is brought into contact with the skin, the first component is dissolved due to moisture, and thus can act on the skin. The moisture as used herein refers to moisture evaporated from the body and/or moisture supplied from the outside. The moisture supplied from the outside may be supplied together with a liquid. Also, the particulates that contain a second component can be brought into contact with the skin by dissolution of the first component. Furthermore, as a result of the fibers containing water, dryness of the skin while the fiber assembly is in contact with the skin is suppressed. The fibers may contain the second component. Also, the fibers may contain a third component that was described above.

The fibers preferably have an average fiber diameter D1 of 600 nm or less, more preferably 200 nm or less, and even more preferably 100 nm or less. This facilitates dissolution of the fibers, and the adhesion to the skin is increased. On the other hand, from the viewpoint of facilitating the support of the particulates, the fibers preferably have an average fiber diameter D1 of 20 nm or more, and more preferably 50 nm or more.

As used herein, the term “average fiber diameter D1” refers to an average value of fiber diameters. Here, the term “fiber diameter” refers to the diameter of a cross section of a fiber, the cross section being perpendicular to the lengthwise direction of the fiber. If the cross section does not have a circular shape, the greatest dimension may be taken as the diameter. Alternatively, a width in a direction perpendicular to the lengthwise direction of a fiber when the fiber assembly is viewed from a direction normal to one of the main surfaces of the fiber assembly may be taken as the fiber diameter. The average fiber diameter D1 is, for example, an average value of diameters measured at arbitrary locations on arbitrarily selected ten fibers of the fiber assembly. In the case where the fibers contain beads, which will be described later, the diameter can be measured by avoiding bead portions.

Here, spindle-shaped bulges (hereinafter, referred to as “beads”) may be formed in the fibers. Unlike the particulates, the beads are formed mainly by the first component that was not sufficiently drawn and thus was not formed into fibers during the process of electrospinning. The beads may contain, together with the first component, the second component and/or the third component blended in the raw material liquid 20. With the beads, the adhesion of the fiber assembly to the skin is improved. Furthermore, with the beads, the duration required for dissolution of the first component increases. Accordingly, it is possible to control the duration of the action of each component contained in the fiber assembly.

There is no particular limitation on the size of the beads. In the case where each component contained in the fiber assembly is caused to act over a long period of time, it is preferable that a plurality of beads are formed on a single fiber. With this configuration, the duration required for dissolution of the fibers can be further increased.

The average diameter D3 of the beads is an average value of the greatest diameters of a plurality of (for example, ten) beads. The greatest diameter of a bead refers to the greatest dimension of the bead at which the outline of the bead is clearly visible when the fiber assembly is viewed from one direction. The greatest diameter of a bead can be determined, for example, in the manner described below. On a SEM micrograph of the fiber assembly, the fiber diameter of a single fiber is measured while moving toward a bead on the single fiber, and a spot where the fiber diameter first reaches two times or more the average fiber diameter D1 is defined as end portion T1 that is one end portion of the bead. Then, the fiber diameter of the same fiber is measured from the opposite side of the same bead while moving toward the bead on the fiber, and a spot where the fiber diameter first reaches two times or more the average fiber diameter D1 is defined as end portion T2 that is the other end portion of the bead. A straight line that connects the end portion T1 and the end portion T2 is drawn, and the greatest length of the bead in a direction perpendicular to the straight line is referred to as the greatest diameter of the bead.

### (Particulates)

The particulates are included in the fiber assembly in a supported state by a plurality of fibers. The particulates contain a second component that is capable of forming a hydrogel. In the case where the fibers contain the second component, the mass proportion R_{2P} of the second component contained in the particulates is greater than the mass proportion R_{2F} of the second component contained in the fibers. The mass proportion R_{2P}-to-mass proportion R_{2F} ratio (R_{2P}/R_{2F}) is, for example, 2 to 20. The mass proportion R_{2P} is, for example, 20 to 80 mass %. Also, from the viewpoint of allowing the second component to easily exhibit its effect, it is preferable that the second component is a main component of the particulates that accounts for 50 mass % or more of the particulates excluding moisture. The particulates may contain the first component and/or a third component.

The second component contained in the particulates form a hydrogel under the presence of moisture. Accordingly, when the fiber assembly is brought into contact with the skin under the presence of moisture, the particulates adhere to the skin and thus can directly act on the skin. From this viewpoint, it is preferable that the particulates contain water together with the second component. This is because when the fiber assembly is brought into contact with the skin, the second component can adhere to the skin without supply of a large amount of moisture. Furthermore, the particulates retain water, and thus dryness of the skin while the fiber assembly is in contact with the skin is also suppressed.

From the viewpoint of the moisture retention or the second component content, the particulates are preferably large. However, if the particulates are too large, interstices are created around the particulates, as a result of which the adhesion of the fiber assembly to the skin easily decreases, and the fiber assembly is easily detached from the skin. From this viewpoint, it is preferable that the average fiber diameter D1 of the fibers and the average particle size D2 of the particulates satisfy the relationship: D1<D2. This allows the particulates to contain a sufficient amount of the second component, and the detachment of the particulates from the fiber structure is more easily suppressed. In particular, it is preferable that the average fiber diameter D1 of the fibers and the average particle size D2 of the particulates satisfy the relationship: D1<D2×1/20, and more preferably satisfy the relationship: D1<D2×1/50.

To be specific, the average particle size D2 of the particulates is preferably 0.2 to and more preferably 0.5 to 10 μm. Accordingly, the particulates are not easily detached, and the amount of the second component contained in the particulates can be increased.

The average particle size D2 of the particulates is an average value of the greatest diameters of a plurality of (for example, ten) particulates of the fiber assembly. The greatest diameter of a particulate refers to the greatest dimension of the particulate at which the outline of the particulate is clearly visible when the fiber assembly is viewed from one direction.

The particulates are required to be incorporated as much as possible in the fiber assembly from the viewpoint of allowing the particulates to easily act on the skin. For example, the mass proportion of the particulates to the fiber assembly is preferably 5 to 40 mass %, and more preferably 10 to 25 mass %. The particulates are supported by a plurality of fibers, and thus are not easily detached from the fiber assembly. Accordingly, the particulates can be incorporated in the fiber assembly in a proportion described above.

### EXAMPLES

Hereinafter, the present invention will be described in further detail by way of examples. However, it is to be noted that the present invention is not limited to the examples given below.

### Example 1

### (1) Preparation of Raw Material Liquid

A raw material liquid (with a viscosity at a shear rate of 1 s⁻¹ of 10.5 Pa·s) was obtained by mixing and dissolving, in ultrapure water, sodium hyaluronate (Na hyaluronate) and collagen peptide (enzyme-degraded collagen peptide, with an average molecular weight of 2,000) so as to achieve a Na hyaluronate concentration of 1.5 mass % and a collagen peptide concentration of 10 mass %.

### (2) Formation of Fiber Assembly

A fiber assembly was obtained by electrospinning the obtained raw material liquid with an applied voltage of 45 kV, the fiber assembly containing fibers having an average fiber diameter D1 of 60 nm and particulates having an average particle size D2 of about 1 μm. A scanning electron microscope (SEM) micrograph of the obtained fiber assembly is shown in FIG. 2. FIG. 2 is a micrograph capturied from one of the main surfaces of the fiber assembly at a magnification of 5,000 times.

As can be seen from FIG. 2, in the obtained fiber assembly, at least a portion of the particulates are supported by a plurality of fibers. That is, the plurality of fibers are bonded to the surface of particulates so as to retain the particulates. On the other hand, the fibers and the particulates are in point contact or line contact, and thus the particulates have a large exposed area.

### (3) Determination of Mass Proportions of Components in Fiber Assembly

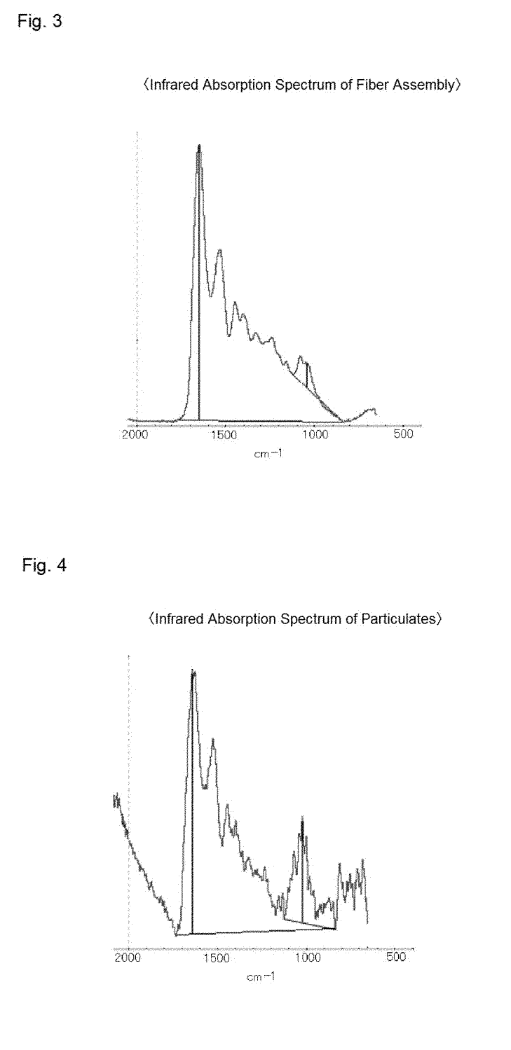

An infrared absorption spectrum of the entire fiber assembly obtained was acquired by a KBr method using a microscopic infrared absorption measurement apparatus (Nicolet 6700 available from ThermoFisher Scientific, Inc.) (FIG. 3). Also, three particulates having a particle size of 2 μm were taken out from the fiber assembly with the use of a manipulator (AXIS-PRO available from Micro Support, Co., Ltd.), and were placed on a KBr plate. After that, an infrared absorption spectrum was acquired in the same manner (FIG. 4).

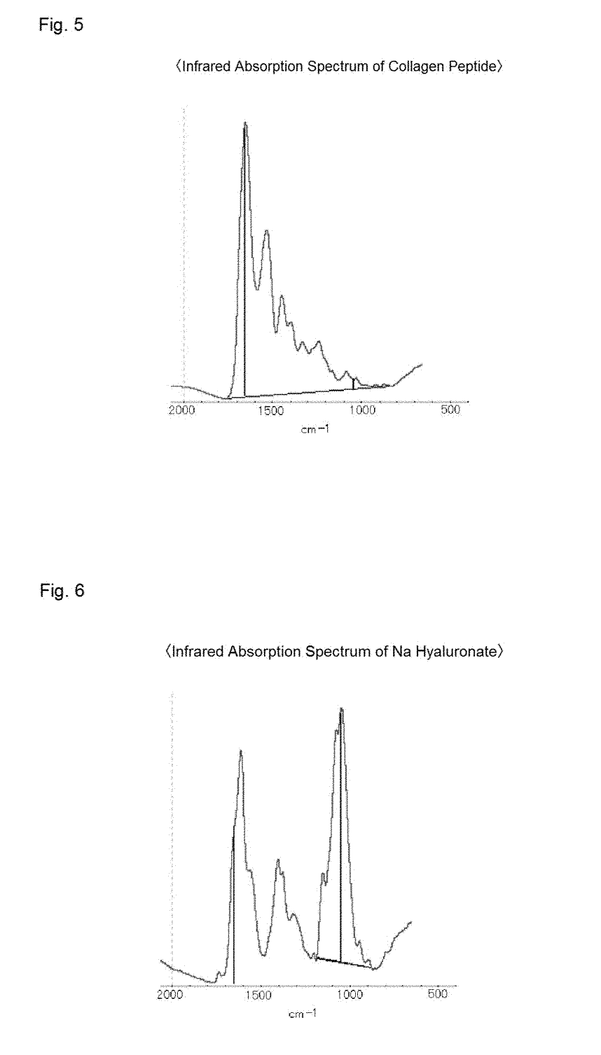

Meanwhile, the above-described collagen peptide and Na hyaluronate powders were placed on KBr plates, respectively, and infrared absorption spectrums that serve as criteria (reference spectrums) were acquired in the same manner as described above. The infrared absorption spectrum of collagen peptide is shown in FIG. 5, and the infrared absorption spectrum of Na hyaluronate is shown in FIG. 6. Collagen peptide had a characteristic absorption peak at 1650 cm⁻¹, and Na hyaluronate had a characteristic absorption peak at 1050 cm⁻¹.

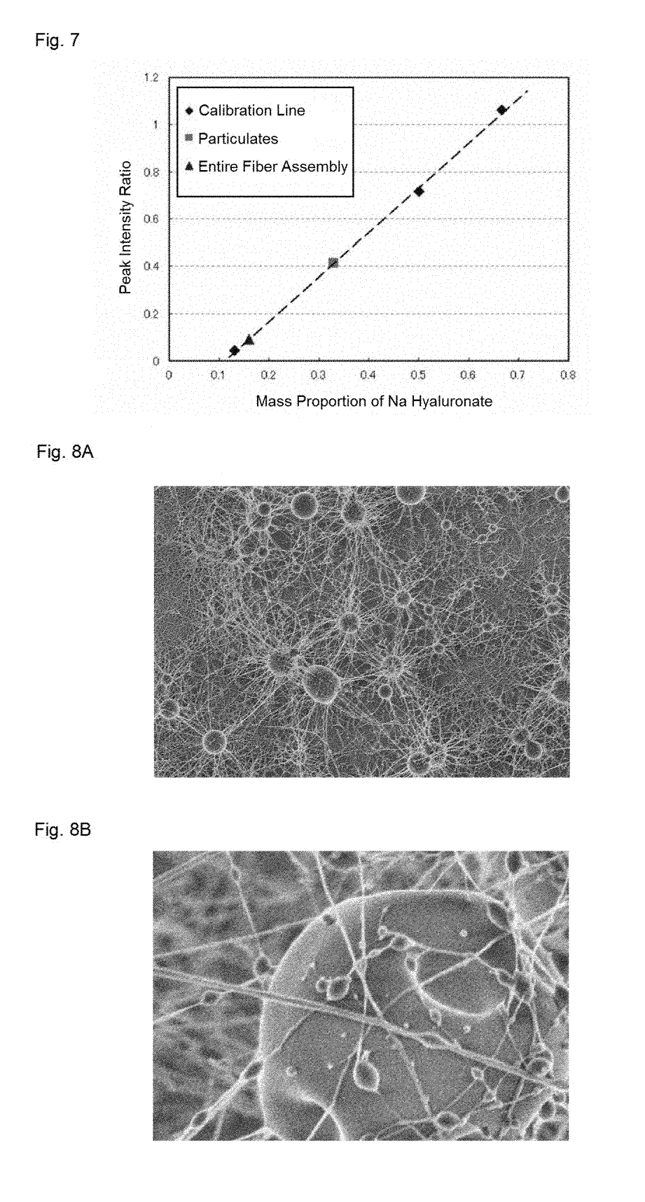

Furthermore, aqueous solutions were prepared by mixing collagen peptide and Na hyaluronate with water so as to achieve a mass ratio of collagen peptide to Na hyaluronate of 67:33, 50:50 and 13:87. Each aqueous solution was applied onto an aluminum foil and dried so as to form a thin film. Then, an infrared absorption spectrum was acquired by using a reflection method under the same conditions as described above. From the infrared absorption spectrum thus acquired and the reference spectrums, a peak intensity ratio was calculated at each of the above-described absorption peaks, and the obtained peak intensity ratios and the mass proportion of Na hyaluronate in the thin film were plotted on a graph. Then, as shown in FIG. 7, a calibration line was drawn based on the plotted points, and the relationship between the peak intensity ratio and the mass proportion of Na hyaluronate was estimated.

The peak intensity ratio calculated from the infrared absorption spectrum of the particulates and the reference spectrums was plotted on the above graph, and the mass proportion R_{2P} of Na hyaluronate contained in the particulates was determined and found to be about 33 mass %. Amass proportion R₂ of Na hyaluronate contained in the entire fiber assembly was determined in the same manner and found to be about 15 mass %. From the calculation results and the mass proportion of the particulates to the fiber assembly, it can be seen that the mass proportion R_{2P} of Na hyaluronate (the second component) contained in the particulates is greater than the mass proportion R_{2F} of Na hyaluronate contained in the fibers. However, it is considered that the actual proportion of the second component in the particulates is greater than the above value calculated from the infrared absorption spectrums because fibers adhering to the particulates or fibers that are present around the particulates are also taken out when the particulates are taken out.

Also, from the above conclusion that the mass proportion R_{2P} of Na hyaluronate contained in the particulates is greater than the mass proportion R₂ of Na hyaluronate contained in the entire fiber assembly, it can be said that the mass proportion R_{2F} of Na hyaluronate contained in the fibers is smaller than the mass proportion R₂ of Na hyaluronate contained in the entire fiber assembly. That is, the mass proportion R_{2F} of Na hyaluronate contained in the fibers is less than 15 mass % (<mass proportion R₂), and the remainder (85 mass % or more) of the fibers is collagen peptide. That is, the main component (the component that accounts for 50 mass % or more of the fibers) of the fibers is collagen peptide.

### Example 2

A fiber assembly was obtained by preparing a raw material liquid (with a viscosity at a shear rate of 1 s⁻¹ of 1 Pa·s) in the same manner as in Example 1, except that Na hyaluronate was mixed and dissolved in ultrapure water so as to achieve a Na hyaluronate concentration of 1 mass %.

SEM micrographs of the obtained fiber assembly are shown in FIGS. 8A and 8B. FIG. 8A is a micrograph captured from one of the main surfaces of the fiber assembly at a magnification of 10,000 times, and FIG. 8B is a micrograph obtained by capturing the same portion as that of FIG. 8A at a magnification of 50,000 times. As can be seen from FIGS. 8A and 8B, in the obtained fiber assembly, at least a portion of the particulates are supported by a plurality of fibers. The fibers had an average fiber diameter D1 of 60 nm, and the particulates had an average particle size D2 of about 1 μm. Also, fibers containing a plurality of beads (average diameter D3: 150 nm) were also observed.

Furthermore, as in Example 1, it was confirmed that collagen peptide accounts for 50 mass % or more of the fibers, and that the mass proportion R_{2P} of Na hyaluronate contained in the particulates is greater than the mass proportion R_{2F} of Na hyaluronate contained in the fibers.

### Example 3

A fiber assembly was obtained by preparing a raw material liquid (with a viscosity at a shear rate of 1 s⁻¹ of 1 Pa·s) in the same manner as in Example 1, except that sodium alginate and collagen peptide were mixed and dissolved in ultrapure water so as to achieve a sodium alginate concentration of 1 mass % and a collagen peptide concentration of 20 mass %

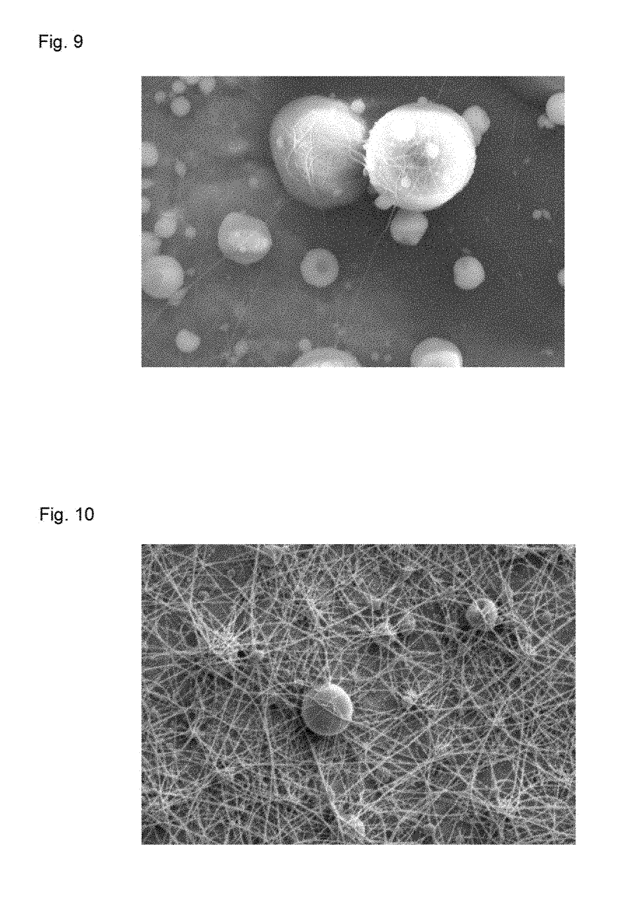

A SEM micrograph of the obtained fiber assembly is shown in FIG. 9. FIG. 9 is a micrograph captured from one of the main surfaces of the fiber assembly at a magnification of 5,000 times. As can be seen from FIG. 9, in the obtained fiber assembly, at least a portion of the particulates are supported by a plurality of fibers. The fibers had an average fiber diameter D1 of 60 nm, and the particulates had an average particle size D2 of about 2 μm.

Furthermore, as in Example 1, it was confirmed that collagen peptide accounts for 50 mass % or more of the fibers, and that the mass proportion R_{2P} of sodium alginate contained in the particulates is greater than the mass proportion R_{2F} of sodium alginate contained in the fibers.

### Example 4

A fiber assembly was obtained by preparing a raw material liquid (with a viscosity at a shear rate of 1 s⁻¹ of 13.1 Pa·s) in the same manner as in Example 1, except that Na hyaluronate and collagen peptide were mixed and dissolved in a phosphoric acid buffer solution (with a pH of 7.4 and a concentration of 10 mM) so as to achieve a Na hyaluronate concentration of 1.5 mass % and a collagen peptide concentration of 10 mass %. The phosphoric acid buffer solution was prepared by dissolving, in ultrapure water, a predetermined amount of pH adjusting agents (sodium dihydrogen phosphate dihydrate and disodium hydrogen phosphate).

A SEM micrograph of the obtained fiber assembly is shown in FIG. 10. FIG. 10 is a micrograph captured from one of the main surfaces of the fiber assembly at a magnification of 5,000 times. As can be seen from FIG. 10, in the obtained fiber assembly, at least a portion of the particulates are supported by a plurality of fibers. The fibers had an average fiber diameter D1 of 60 nm, and the particulates had an average particle size D2 of about 1.5 μm.

Furthermore, as in Example 1, it was confirmed that collagen peptide accounts for 50 mass % or more of the fibers, and that the mass proportion R_{2P} of Na hyaluronate contained in the particulates is greater than the mass proportion R_{2F} of Na hyaluronate contained in the fibers.

### INDUSTRIAL APPLICABILITY

With the method for manufacturing a fiber assembly according to the present invention, a fiber assembly that contains fibers and particulates supported by the fibers can be manufactured in a very simple process. Accordingly, the method for manufacturing a fiber assembly according to the present invention is suitable for manufacturing, in addition to sheets for application to the skin, functional fiber assemblies used in various types of applications. Also, because the fiber assembly according to the present invention contains fibers and particulates supported by the fibers, it is possible to increase the exposed area of the particulates while suppressing detachment of the particulates.

### DESCRIPTION OF REFERENCE SIGNS

10: Electrospinning Apparatus
11: Discharging Unit
12: Target
13: Conveyor Belt
14: Voltage Application Apparatus
15: Counter Electrode
16: First Supporting Unit
17: Second Supporting Unit
18: Pipe
20: Raw Material Liquid

1. A method for manufacturing a fiber assembly comprising:
a preparation step of preparing a raw material liquid that contains a water-soluble first component, a second component that is capable of forming a hydrogel, and water; and
an electrospinning step of forming fibers that contain the first component as a main component and particulates that are supported by a plurality of the fibers and contain the second component from the raw material liquid by an electrospinning method,
wherein in a case where the fibers contain the second component, a mass proportion of the second component contained in the particulates is greater than a mass proportion of the second component contained in the fibers.

2. The method for manufacturing a fiber assembly in accordance with claim 1,
wherein an average fiber diameter D1 of the fibers and an average particle size D2 of the particulates satisfy the relationship: D1<D2.

3. The method for manufacturing a fiber assembly in accordance with claim 1,
wherein the fibers have an average fiber diameter D1 of 600 nm or less.

4. The method for manufacturing a fiber assembly in accordance with claim 1,
wherein the second component is at least one selected from the group consisting of a hyaluronic acid salt, a hyaluronic acid derivative, a water-soluble alginic acid salt, and an alginic acid derivative, and
the particulates contain the water together with the second component.

5. The method for manufacturing a fiber assembly in accordance with claim 1,
wherein the first component is any of collagens.

6. The method for manufacturing a fiber assembly in accordance with claim 1,
wherein the first component is any of collagens,
the second component is at least one selected from the group consisting of a hyaluronic acid salt, a hyaluronic acid derivative, a water-soluble alginic acid salt, and an alginic acid derivative, and
the raw material liquid further contains a third component other than the first component and the second component.

7. The method for manufacturing a fiber assembly in accordance with claim 6,
wherein the third component is a pH adjusting agent.

8. A fiber assembly comprising:
fibers that contain a water-soluble first component as a main component; and
particulates that contain a second component that is capable of forming a hydrogel,
wherein at least a portion of the particulates are supported by a plurality of the fibers, and
in a case where the fibers contain the second component, a mass proportion of the second component contained in the particulates is greater than a mass proportion of the second component contained in the fibers.

9. The fiber assembly in accordance with claim 8,
wherein an average fiber diameter D1 of the fibers and an average particle size D2 of the particulates satisfies the relationship: D1<D2.

10. The fiber assembly in accordance with claim 8,
wherein the fibers have an average fiber diameter D1 of 600 nm or less.

11. The fiber assembly in accordance with claim 8,
wherein the second component is at least one selected from the group consisting of a hyaluronic acid salt, a hyaluronic acid derivative, a water-soluble alginic acid salt, and an alginic acid derivative, and
the particulates contain water together with the second component.

12. The fiber assembly in accordance with claim 8,
wherein the first component is any of collagens,
the second component is at least one selected from the group consisting of a hyaluronic acid salt, a hyaluronic acid derivative, a water-soluble alginic acid salt, and an alginic acid derivative, and
at least either of the fibers and the particulates further contain a third component other than the first component and the second component.

13. The fiber assembly in accordance with claim 12,
wherein the third component is a pH adjusting agent.
